(19) United States
(12) Patent Application Publication
(10) Pub. No.: US 2022/0361944 A1
(43) Pub. Date: Nov. 17, 2022

(54) Energy Delivery System, Method and Device

(71) Applicant: BOSTON SCIENTIFIC SCIMED, INC., Maple Grove, MN (US)

(72) Inventors: Louis McNern, Donegal (IE); Kevin McEvilly, Galway (IE); Claudia Akhimein, Shannon (IE); Aaron Rosenthal, Medway, MA (US); Alexander McLane, Wellesley, MA (US)

(73) Assignee: BOSTON SCIENTIFIC SCIMED, INC., Maple Grove, MN (US)

(21) Appl. No.: 17/746,236

(22) Filed: May 17, 2022

Related U.S. Application Data
(60) Provisional application No. 63/189,568, filed on May 17, 2021.

(51) Int. Cl. A61B 18/14

(52) U.S. Cl. CPC A61B 18/1492; A61B 2018/00613

(57) ABSTRACT

A system and method for delivering irreversible electroporation treatment to a selected tissue includes introducing an endoscope into a position adjacent the selected tissue, and advancing first and second probes having electrodes at their distal ends. One probe may be inserted into the lesion or tumor and remain in place while the other probe is moved around the tumor, with activation of the electrodes occurring at each location of the probes.

### CROSS-REFERENCE TO RELATED APPLICATIONS

This application claims priority to U.S. Provisional Application Ser. No. 63/189,568 filed May 17, 2021, the entirety of which is incorporated herein by reference.

### TECHNICAL FIELD

The disclosure pertains to medical devices and more particularly to energy delivery systems and devices such as irreversible electroporation devices, and methods for using such medical devices.

### BACKGROUND

A number of energy based ablation modalities are used to treat biological tissue such as abnormal tissue including tumors. The various modalities known each have shortcomings. For example, cryoablation and thermal ablation can be difficult to control in spatial extent, particularly with vascularized tissue, and can be non-specific, impairing the post-ablation healing process in the vicinity of tumor removal. Chemical ablation can have serious and sometimes systemic side effects.

Electroporation has been used in various forms to treat targeted tissue. Electroporation operates by applying electrical pulses that cause cell membranes to alter, creating pores. Above a first threshold electrical field, the cell membranes begin to form pores. After removal of the electrical field, the induced pores close. This reversible electroporation may be used in conjunction with the infusion of drugs or other agents which pass through reversibly created pores, and it is the drug or agent which causes cell death. Above a second, higher threshold field, those pores can become irreversible, leading to cell death. Thus there are two forms of electroporation, reversible electroporation and irreversible electroporation (IRE).

IRE can be used in addition to or instead of surgery, and used in some cases in complement to chemotherapy and radiation therapies when tumors cannot be removed surgically. It is also an option for patients who are not candidates for ablation therapies such as cryoablation, microwave ablation or radiofrequency ablation. Treatment areas include but not limited to prostate, liver, lung, kidney and pancreas. One benefit of IRE is the fact it can be used on tumors which are anatomically neighboring blood cells, bile ducts and the colon.

New and different approaches to biological tissue destruction using energy delivery systems and devices are desired. In particular, new approaches to using irreversible electroporation for tissue ablation are desired.

### SUMMARY

This disclosure provides design, material, manufacturing method, and use alternatives for medical devices. An example method for delivering irreversible electroporation treatment to a selected tissue comprises introducing an endoscope into a position with a distal end of the endoscope adjacent the selected tissue, the endoscope having a working channel, wherein a first probe is disposed within the working channel, the first probe including at least a first electrode disposed at a distal end thereof, inserting the first probe into the selected tissue at a first location, advancing a second probe along an outer surface of the endoscope, the second probe having at least a second electrode disposed at a distal end thereof, inserting the second probe into the selected tissue at a second location spaced apart from the first location, activating the first and second electrodes, moving the second probe to at least a third location spaced apart from the first and second locations, and activating the first and second electrodes.

Alternatively or additionally to the embodiment above, moving the second probe to the third location is performed while maintaining the first probe in the first location.

Alternatively or additionally to any of the embodiments above, the method further comprises moving the second probe to additional locations within the selected tissue and activating the first and second electrodes at each location of the second probe, the additional locations being spaced apart from the first, second, and third locations.

Alternatively or additionally to any of the embodiments above, the first location is in a center region of the selected tissue and the second, third, and additional locations are around a perimeter of the selected tissue.

Alternatively or additionally to any of the embodiments above, moving the second probe to additional locations includes moving the second probe sequentially to the additional locations around the perimeter of the selected tissue, and activating the first and second probes at leach new location of the second probe.

Alternatively or additionally to any of the embodiments above, the endoscope is moveable proximally and distally while the first probe remains in the first location.

Alternatively or additionally to any of the embodiments above, the second probe is disposed within a sleeve disposed adjacent to the outer surface of the endoscope.

Alternatively or additionally to any of the embodiments above, the sleeve is fixed to the outer surface of the endoscope.

Alternatively or additionally to any of the embodiments above, moving the second probe includes moving the endoscope to position the second probe at the third location.

Alternatively or additionally to any of the embodiments above, the endoscope includes an imaging device and angulation controls, wherein moving the endoscope includes using the imaging device and angulation controls to move the second probe to the third location.

Alternatively or additionally to any of the embodiments above, the second probe is fixed within the sleeve.

Alternatively or additionally to any of the embodiments above, the second probe is moveable relative to the sleeve.

Alternatively or additionally to any of the embodiments above, the first probe includes a fixation element configured to removably secure the first probe within the selected tissue.

Alternatively or additionally to any of the embodiments above, the first and second locations are spaced approximately 0.1 cm to 10.0 cm apart. Alternatively or additionally to any of the embodiments above, the first and second locations are spaced approximately 1.5 cm to 2.0 cm apart.

Alternatively or additionally to any of the embodiments above, the second and third locations are spaced approximately 0.1 cm to 10.0 cm apart, and the first and third locations are spaced approximately 0.1 cm to 10.0 cm apart.

Alternatively or additionally to any of the embodiments above, the endoscope includes a locking member configured to lock the first probe relative to the endoscope.

Another example method for delivering irreversible electroporation treatment to a selected tissue comprises introducing an endoscope into a position with a distal end of the endoscope adjacent the selected tissue, the endoscope having a working channel, wherein a first probe is disposed within the working channel, the first probe including at least a first electrode disposed at a distal end thereof, advancing a second probe along an outer surface of the endoscope, the second probe having at least a second electrode disposed at a distal end thereof, inserting one of the first and second probes into the selected tissue at a first location, inserting remaining probe into the selected tissue at a second location spaced apart from the first location, activating the first and second electrodes, moving the probe disposed at the second location to at least a third location spaced apart from the first and second locations, and activating the first and second electrodes.

Alternatively or additionally to the embodiment above, the first location is in a central region of the selected tissue, the method further comprising moving the probe disposed at the second location sequentially to additional locations around a perimeter of the selected tissue and activating the first and second electrodes at each location of the probe around the perimeter, the additional locations being spaced apart from the first, second, and third locations, wherein moving the probe disposed at the second location to the third location is performed while maintaining the probe inserted into the first location at the first location.

An example system for irreversible electroporation of tissue comprises an endoscope having a working channel, a first probe disposed within the working channel, the first probe including at least a first electrode disposed at a distal end thereof, a second probe disposed within a sleeve disposed on an outer surface of the endoscope, the second probe having at least a second electrode disposed at a distal end thereof, and wherein the second probe is moveable axially and radially relative to the first probe.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A illustrates an example medical device assembly disposed adjacent a selected tissue;

FIG. 1B illustrates alternative attachment members on a probe;

FIG. 2 is a block flow diagram of an example method; and

FIG. 3 illustrates positions for probes A and B during a treatment method.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

As described in U.S. Pat. No. 6,010,613, a transmembrane potential in the range of about one volt is needed to cause reversible electroporation, however the relationship between pulse parameters such as timing and duration and the transmembrane potential required for reversible electroporation remains an actively investigated subject. The required field may vary depending on characteristics of the cells to be treated. At a macro level, reversible electroporation requires a voltage in the level of hundreds of volts per centimeter, with irreversible electroporation requiring a still higher voltage. As an example, when considering in vivo electroporation of liver tissue, the reversible electroporation threshold field strength may be about 360 V/cm, and the irreversible electroporation threshold field strength may be about 680 V/cm, as described in U.S. Pat. No 8,048,067. Generally speaking, a plurality of individual pulses are delivered to obtain such effects across the majority of treated tissue; for example, 2, 4, 8, 16, or more pulses may be delivered.

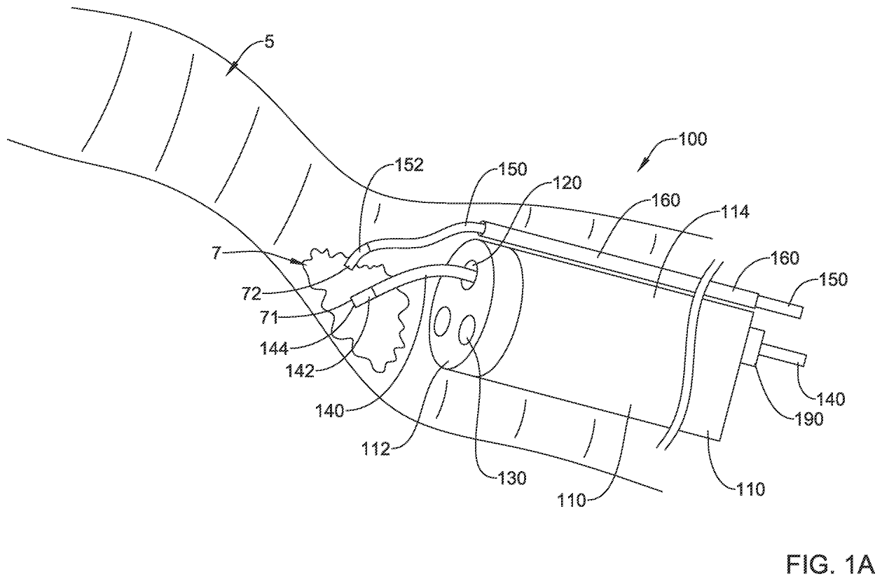

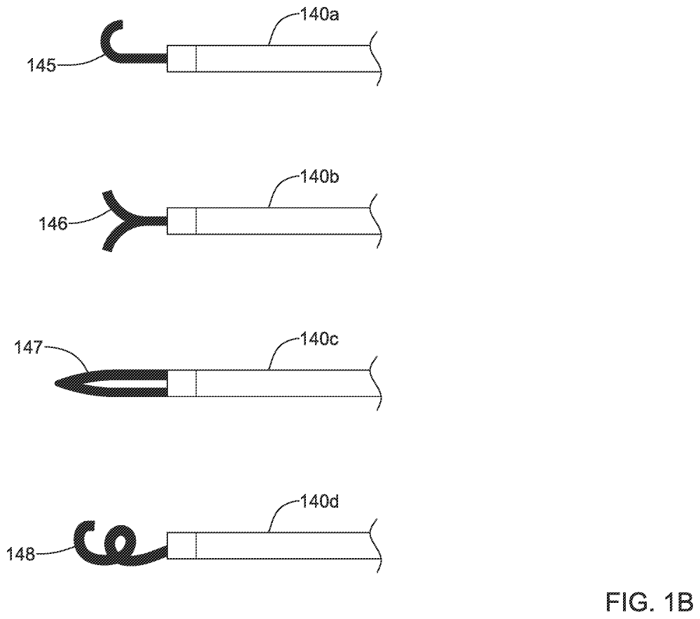

The field for electroporation has typically been applied by delivering a series of individual pulses each having a duration in the range of tens to hundreds of microseconds. For example, U.S. Pat. No. 8,048,067 describes a series of eight 100 microsecond pulses delivered at 1 second intervals. The '067 patent describes analysis and experiments performed to illustrate that the area between lines 20 and 30 in FIG. 1 actually exists, and that a non-thermal IRE method can be achieved.

The tissue membrane does not return instantaneously, from a porated state. As a result, the application of pulses close together in time can have a cumulative effect as described, for example, in U.S. Pat. No. 8,926,606. In addition, a series of pulses can be used to first porate a cell membrane and then move large molecules through generated, reversible pores, as described in U.S. Pat. No. 7,608,275.

Prior irreversible electroporation (IRE) procedures were most commonly performed using percutaneous and/or laparoscopic access to the target site, which require anesthesia that increases post procedure recovering time and carry certain risks including bleeding, infection and damage to organs in the abdomen. An advancement in IRE treatment involves the ability for it to be delivered in an endoscopy setting performed by a gastroenterologist, rather than by percutaneous or laparoscopic procedures performed by a surgeon.

An electroporation assembly may be delivered endoscopically, utilizing the minimally invasive benefits of endoscopic treatment while also eliminating the associated risks with percutaneous and/or laparoscopic methods of delivering IRE treatment. The assembly allows physicians to deliver IRE treatment by introducing at least a first probe through the working channel of the endoscope and at least one second probe through an outer sleeve. It is understood that additional second probes and outer sleeves or external working channels may be provided on an endoscope for performing a tissue ablation procedure.

FIG. 1A illustrates a tissue ablation assembly 100 inserted through a portion of a gastrointestinal (GI) tract, e.g., the colon, 5 to a position adjacent a selected tissue, e.g., a lesion or tumor, 7 to be treated. Although the GI tract is described herein, it is understood that a tissue ablation system may be deliverable to other anatomical structures in a human or animal body for a treatment procedure and may be utilized with an endoscope, bronchoscope, duodenoscope, gastroscope, colonoscope, ureteroscope, tubing, catheter, or the like. The assembly 100 may be delivered to a selected tissue via an endoscope 110 with at least one working channel 120 and an imaging device 130 disposed at the distal end 112 of the endoscope 110. The endoscope 110 may be positioned with its distal end 112 adjacent the selected tissue 7. Two probes (A and B) may be deployable through the endoscope 110. In the configuration illustrated in FIG. 1A, probe A 140 with a first electrode 142 on its distal end extends through the working channel 120 and is inserted into a first location 71 of the selected tissue 7. Probe B 150 with a second electrode 152 on its distal end extends along the outer surface 114 of the endoscope 110, and is inserted into a second location 72 of the selected tissue 7. In some examples, probe B 150 may have at least a partially flexible shaft and may be deployed through a sleeve 160 disposed adjacent the outer surface 114 of the endoscope 110. The flexible shaft on probe B allows the probe to be moved and inserted into various locations within the selected tissue. The first location 71 and the second location 72 are spaced apart. In the example illustrated in FIG. 1A, the first location 71 is in the center region of the selected tissue 7 and the second location 72 is adjacent the perimeter of the selected tissue 7.

Probe A 140 may be disposed through the working channel 120 such that the endoscope may move proximally and distally relative to probe A 140. In some examples, probe A may have at least a partially flexible shaft and the endoscope may also move radially relative to probe A 140 while the probe remains in position with the selected tissue. In other examples, probe A 140 may be fixed within the endoscope 110. In some examples the endoscope 110 may include a locking member 190 configured to lock probe A 140 relative to the endoscope 110 and/or probe B 150. The locking member 190 may lock probe A 140 axially, rotationally, or both. In some examples, a locking member 190 may be configured to lock probe B 150 relative to the endoscope 110 and/or probe A. The locking member 190 may lock probe B 150 axially, rotationally, or both. In some embodiments, a locking member 190 may lock both probes A and B 140, 150 simultaneously, or independently, axially, radially, rotationally, or any combination thereof, relative to the endoscope 110. The sleeve 160 may be fixed to the outer surface 114 of the endoscope 110 along at least a portion of the endoscope. In some examples, the sleeve 160 may be moveable relative to at least the distal region of the endoscope. For example, the sleeve 160 may be configured to move proximally, distally and/or around at least part of the circumference of the endoscope 110, allowing probe B to move around the perimeter of the endoscope 110 to various positions along the perimeter of the selected tissue 7. In some examples, probe B 150 may be fixed within the sleeve 160. Regardless of whether the sleeve 160 is fixed or moveable relative to the endoscope 110, probe B 150 is configured to move completely independently relative to probe A 140, including axially and radially. It is also understood that probe A 140 may move completely independently relative to probe B 150, including axially, radially, or rotationally.

In some embodiments, probe A 140 may include a fixation element 144 attached to its distal end. The fixation element 144 may be configured to releasably hold the first electrode 142 at a fixed position within the selected tissue 7 during treatment. As shown in FIG. 1B, the fixation element on probe A 140a, 140b, 140c, 140d (collectively 140) may be a hook 145, one or more tines 146, a sharp point 147, a coil 148, or any other structure configured to retain the distal end of probe A 140 in contact with the selected tissue 7.

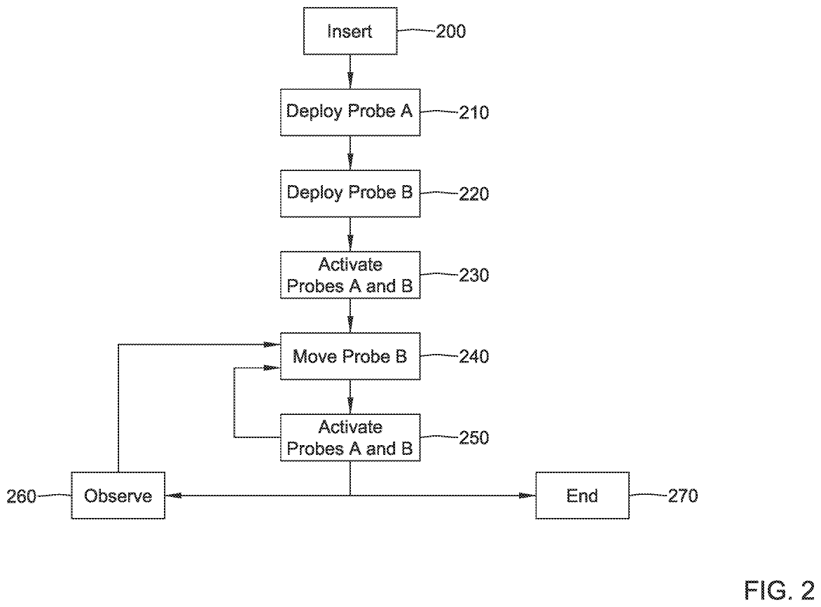

FIG. 2 is a block flow diagram illustrating a method of delivering irreversible electroporation treatment to a selected tissue. Insertion 200 may make use of an existing lumen or channel of the patient (such as the colon), or may comprise piercing tissue with an instrument. Once inserted to a desired location adjacent a treatment location such as a tumor, the treatment apparatus may deploy probe A at step 210, followed by deployment of probe B at step 220, where each probe includes at least one electrode at a distal end thereof. Therapy is then delivered by activating the electrodes on probes A and B, as indicated at step 230. The therapy may be, for example, an IRE therapy in which monophasic or biphasic (or triphasic or other multiphasic) electrical output is generated with relatively high amplitudes (yielding fields of over 600 V/cm, for example) and short pulse widths (for example in the range of 0.1 to 100 microseconds) at a relatively lower duty cycle (such as 1 to 100 Hz—such as a duty cycle of less than 0.1%), which may avoid thermal heating to yield predominantly IRE therapy. After therapy delivery at step 230, probe B is moved to a new location, at step 240. Therapy is delivered again at step 250 by activating the electrodes on probes A and B. The loop of moving probe B at step 240 and activating probes A and B at step 250 may be repeated until the desired treatment is completed. The treatment areas may slightly overlap to ensure the treatment is delivered to the complete surface area of the tumor.

In some examples a set quantity of therapy steps and adjustments 240/250 may be performed and the method ends by exiting the loop 240/250, proceeding to the end block 270. In other examples, after one or more therapy steps 250, the method engages an observation step 260, in which one or more observable features are quantities or checked to determine progress or status of the therapy. For example, the observable features may refer to temperature, impedance, and/or an imaging modality such as a CT image. In some examples, impedance may be checked between the two probe electrodes. It should be understood that as therapy progresses, cell death may occur, releasing intercellular fluid into the extracellular matrix and reducing impedance as cell death occurs, making impedance a useful observation. Also, as therapy progresses, temperature may be checked to ensure that temperatures as measured using, for example a temperature sensor on the endoscope or a temperature sensor on a separate probe, is in a desired range. For example, as cell death occurs, local temperature may increase more greatly as local impedance drops and current flows increase at a given voltage, making temperature a useful measure of status. An image may be used as well to determine the status of the tumor or lesion. After observation 260, an adjustment 240 may be made if desired or therapy 250 may resume. If observation 260 shows satisfactory completion of treatment, the method may go to the end block 270 if desired.

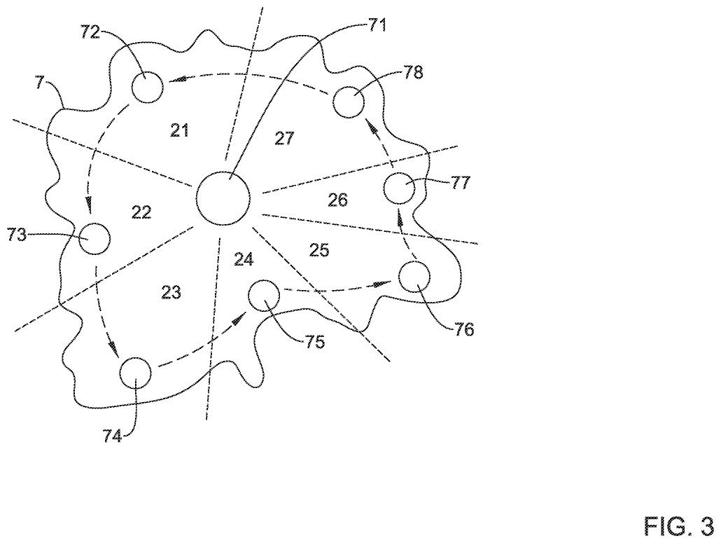

In the method illustrated in FIG. 1A, probe A is deployed in a first location 71 in the center region of the selected tissue 7, and probe B is deployed in the second location 72 adjacent the perimeter of the selected tissue 7. After activating the electrodes on probe A and probe B in step 230 of the method, probe B is moved to a third location in step 240, as shown in FIG. 2. As discussed above, the method may involve the sequential steps of moving probe B and activating probes A and B in a repeating loop until the desired treatment is completed, with probe B being moved so the treatment regions overlap slightly. FIG. 3 illustrates locations for probe B in an example method. Probe A may be inserted into the central region 71 of the selected tissue 7, and remain in this location for the duration of the treatment as probe B is moved. Probe B may be initially inserted into location 72 at the periphery of the selected tissue 7. Activation of probes A and B provides electroporation therapy to a zone 21 between locations 71 and 72. Probe B may then be moved to location 73, followed by activation of probes A and B to provide electroporation therapy to zone 22. Probe B may then be moved to location 74, followed by electroporation treatment of zone 23; then moved to location 75 followed by electroporation treatment of zone 24; then moved to location 76 followed by electroporation treatment of zone 25; then moved to location 77 followed by electroporation treatment of zone 26; then moved to location 78 followed by electroporation treatment of zone 27. The sequential movement of probe B may allow for sequential treatment around the selected tissue. In FIG. 3, the zones 21, 22, 23, 24, 25, 26, 27 are illustrated as having borders denoted by dotted lines, however it will be understood that the zones may slightly overlap to ensure complete treatment of the selected tissue. The method may ensure no healthy tissue is impacted by the electroporation energy because probe B is inserted within the selected tissue around the perimeter, thereby creating an arc of current between probe A at location 71 and probe B at the various locations 72, 73, 74, 75, 76, 77, 78. The arcs of current provide treatment to the zones 21, 22, 23, 24, 25, 26, 27. Movement of probe B may be achieved by moving the endoscope 110, using angulation controls and the imaging device 130 on the endoscope 110. In other examples, probe B 150 may be inserted into the first location 71 and probe A 140 may be inserted into the second location 72 and then moved sequentially to the other locations 72, 73, 74, 75, 76, 77, 78.

The number and position of locations in which probe B is placed may be adjusted according to the outer geometry and topography of the selected tissue 7. For example, if the selected tissue has large ridges, the locations where probe B is deployed may be closer together as compared to a flatter selected tissue in which the locations may be farther apart. In general, the locations for probe B may be spaced apart by about 0.1 cm to about 10.0 cm, or about 1.0 cm to about 3.0 cm. In some examples, the locations for probe B may be spaced apart by about 1.5 cm to about 2.0 cm. Additionally, the distance between the location for probe A and each location for probe B may be about 1.0 cm to about 3.0 cm, or about 1.5 cm to about 2.0 cm. For tumors larger than 4 cm, probe A may be moved to multiple locations. For example, probe A may be placed in a first location and probe B may be moved around a section of the selected tissue perimeter adjacent probe A, with activation of the electrodes on the probes A and B at each location of probe B. Then probe A may be moved to a second location and probe B may be moved around another section of the selected tissue perimeter adjacent probe A. The series of movements and activation of probes A and B may be continued until the entire selected tissue has been treated.

In some examples, the therapy may include injection of a fluid to enhance or modify effectiveness or spatial effects of an applied electrical therapy, or may instead include injection of an ablative fluid such as a fluid having limited caustic effects, or cooling or heating effects. The therapy may include a thermal treatment, which may incorporate somewhat lower pulse amplitudes (fields of less than 600 V/cm, for example) at longer pulse widths (for example, 10 microseconds to 100 milliseconds) at a relatively higher duty cycle (such as by application of the pulses at a frequency of 10 Hz to 100 kHz, in some examples to yield a duty cycle of greater than 0.1%). For example, saline may be injected to reduce local tissue impedance, increasing current flow for a given output voltage, such that both an electrical output is delivered as well as the fluid. Some examples may use both IRE and thermal ablation from a single output waveform by increasing pulse width and/or the duty cycle of IRE outputs to cause thermal effects.

In at least some embodiments, portions or all of the assembly 100 (and variations, systems or components thereof disclosed herein) may be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the assembly 100, particularly probe A and probe B. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the assembly 100 (and variations, systems or components thereof disclosed herein) to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A method for delivering irreversible electroporation treatment to a selected tissue comprising:
introducing an endoscope into a position with a distal end of the endoscope adjacent the selected tissue, the endoscope having a working channel, wherein a first probe is disposed within the working channel, the first probe including at least a first electrode disposed at a distal end thereof;
inserting the first probe into the selected tissue at a first location;
advancing a second probe along an outer surface of the endoscope, the second probe having at least a second electrode disposed at a distal end thereof;
inserting the second probe into the selected tissue at a second location spaced apart from the first location;
activating the first and second electrodes;
moving the second probe to at least a third location spaced apart from the first and second locations; and
activating the first and second electrodes.

2. The method of claim 1, wherein moving the second probe to the third location is performed while maintaining the first probe in the first location.

3. The method of claim 2, further comprising moving the second probe to additional locations within the selected tissue and activating the first and second electrodes at each location of the second probe, the additional locations being spaced apart from the first, second, and third locations.

4. The method of claim 3, wherein the first location is in a center region of the selected tissue and the second, third, and additional locations are around a perimeter of the selected tissue.

5. The method of claim 4, wherein moving the second probe to additional locations includes moving the second probe sequentially to the additional locations around the perimeter of the selected tissue, and activating the first and second probes at leach new location of the second probe.

6. The method of claim 1, wherein the endoscope is moveable proximally and distally while the first probe remains in the first location.

7. The method of claim 6, wherein the second probe is disposed within a sleeve disposed adjacent to the outer surface of the endoscope.

8. The method of claim 7, wherein the sleeve is fixed to the outer surface of the endoscope.

9. The method of claim 7, wherein moving the second probe includes moving the endoscope to position the second probe at the third location.

10. The method of claim 9, wherein the endoscope includes an imaging device and angulation controls, wherein moving the endoscope includes using the imaging device and angulation controls to move the second probe to the third location.

11. The method of claim 7, wherein the second probe is fixed within the sleeve.

12. The method of claim 7, wherein the second probe is moveable relative to the sleeve.

13. The method of claim 1, wherein the first probe includes a fixation element configured to removably secure the first probe within the selected tissue.

14. The method of claim 1, wherein the first and second locations are spaced approximately 0.1 cm to 10.0 cm apart.

15. The method of claim 14, wherein the first and second locations are spaced approximately 1.5 cm to 2.0 cm apart.

16. The method of claim 14, wherein the second and third locations are spaced approximately 0.1 cm to 10.0 cm apart, and the first and third locations are spaced approximately 0.1 cm to 10.0 cm apart.

17. The method of claim 1, wherein the endoscope includes a locking member configured to lock the first probe relative to the endoscope.

18. A method for delivering irreversible electroporation treatment to a selected tissue comprising:
introducing an endoscope into a position with a distal end of the endoscope adjacent the selected tissue, the endoscope having a working channel, wherein a first probe is disposed within the working channel, the first probe including at least a first electrode disposed at a distal end thereof;
advancing a second probe along an outer surface of the endoscope, the second probe having at least a second electrode disposed at a distal end thereof;
inserting one of the first and second probes into the selected tissue at a first location;
inserting remaining probe into the selected tissue at a second location spaced apart from the first location;
activating the first and second electrodes;
moving the probe disposed at the second location to at least a third location spaced apart from the first and second locations; and
activating the first and second electrodes.

19. The method of claim 18, wherein the first location is in a central region of the selected tissue, the method further comprising moving the probe disposed at the second location sequentially to additional locations around a perimeter of the selected tissue and activating the first and second electrodes at each location of the probe around the perimeter, the additional locations being spaced apart from the first, second, and third locations, wherein moving the probe disposed at the second location to the third location is performed while maintaining the probe inserted into the first location at the first location.

20. A system for irreversible electroporation of tissue comprising:
an endoscope having a working channel;
a first probe disposed within the working channel, the first probe including at least a first electrode disposed at a distal end thereof;
a second probe disposed within a sleeve disposed on an outer surface of the endoscope, the second probe having at least a second electrode disposed at a distal end thereof; and
wherein the second probe is moveable axially and radially relative to the first probe.
